(19) United States
(12) Patent Application Publication
(10) Pub. No.: US 2016/0338791 A1
(43) Pub. Date: Nov. 24, 2016

(54) SURGICAL DRAPE

(71) Applicant: CEDARS-SINAI MEDICAL CENTER, Los Angeles, CA (US)

(72) Inventor: Frederick Smith, Burbank, CA (US)

(73) Assignee: Cedars-Sinai Medical Center, Los Angeles, CA (US)

(21) Appl. No.: 15/114,176

(22) PCT Filed: Feb. 27, 2015
(86) PCT No.: PCT/US15/18056

§ 371 Date: Jul. 26, 2016

Related U.S. Application Data
(60) Provisional application No. 61/946,440, filed on Feb. 28, 2014.
(60) Provisional application No. 62/028,931, filed on Jul. 25, 2014.

(51) Int. Cl. A61B 46/20; A61B 90/00

(52) U.S. Cl. CPC A61B 46/20; A61B 90/39; A61B 2090/3983; A61B 2046/205

(57) ABSTRACT

Some embodiments of the invention include a device for creating a sterile environment when using an o-shaped or a c-shaped multidimensional surgical imaging system. The device may comprise a drape configured to drape over a patient, a cover removably attached to the drape, and an adhesive seam configured to pull apart to disassemble the drape after. The drape may have a puncture configured to engage with a spine reference clamp, a reference frame, each of the puncture and the reference frame positioned along a center line of the drape, and the cover may be removably attached to the drape to cover the puncture and the reference frame.

### CROSS REFERENCE TO RELATED APPLICATIONS

This application claims priority to U.S. Provisional Application No. 61/946,440 filed Feb. 28, 2014, and to U.S. Provisional Application No. 62/028,931 filed Jul. 25, 2014, both titled “Surgical Drape”, and the contents of which are incorporated herein by reference.

### FIELD OF INVENTION

This invention relates to sterile surgical accessories, and in particular, a surgical drape for use with imaging systems.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Certain imaging systems used during operations require sterile drapes to protect the machine from contamination and maintain the sterile field for the patient. Some examples of these types of imaging systems include an O-ARM, C-ARM, CT, MRI, and Body Tom. For example, an O-ARM imaging system is a surgical imaging system that provides intraoperative imaging. The O-ARM system can be used before, during, and after surgery and provides a representation of a patient's anatomy in multiple dimensions in real time in the operating room. An O-ARM system completely encircles the patient. A C-ARM imaging system is similar to an O-ARM imaging system, but it does not fully encircle the patient and instead forms a C shape with the emitter and detector at the ends of the C.

Generally, drapes are used with these types of imaging systems to provide a sterile barrier between the imaging system and patient undergoing surgery. Conventional drapes that are used with the O-ARM or C-ARM systems include drapes that cover or are directly attached to the machine, such as tube drapes. For instance, tube drapes may be wrapped around the entire machine to fully cover the machine's gantry. These drapes therefore require multiple people to position and can be cumbersome to attach. For example, during the positioning of the drape, the drape may become stuck in the gantry door of an O-ARM imaging system, possibly damaging the machine and/or the drape itself. These drapes also do not provide a barrier between the surgeon and other caregivers and the patient undergoing surgery, as they generally do not cover or are not applied to the patient. Accordingly, conventional drapes fail to secure the sterile field.

### SUMMARY OF THE INVENTION

There is a need for a surgical drape for use with an O-ARM, C-ARM, or other imaging systems that can be positioned by a single person, while keeping the patient in a sterile environment. Such a drape would provide a more efficient system to protect the medical equipment from contamination from the patient while undergoing surgery. Additionally, the drape would have the added benefit of isolating the surgical environment of the patient from the caregivers performing surgery. Furthermore, since such a drape could be positioned directly to cover a patient, it may also incorporate reference frames required for the imaging equipment.

The following embodiments and aspects thereof are described and illustrated in conjunction with compositions and methods which are meant to be exemplary and illustrative, not limiting in scope. Some embodiments of the invention include a device for creating a sterile environment when using an o-shaped or c-shaped multidimensional surgical imaging system.

Some embodiments of the device may comprise a drape configured to drape over a patient, a cover removably attached to the drape, and an adhesive seam configured to unseal in order to disassemble the drape into two, three, four or other number of portions after or during use. The drape may have a puncture configured to engage with a spine reference clamp and/or a reference frame, each of the puncture and the reference frame may be positioned along a center line of the drape or in other positions, and the cover may be removably attached to the drape to cover the puncture and the reference frame.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a surgical drape with the patient in a supine position in accordance with various embodiments of the present invention.

FIG. 2 depicts various dimensions and features of the surgical drape in accordance with various embodiments of the present invention.

FIG. 3 depicts various dimensions and features of the cover in accordance with various embodiments of the present invention

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The device of the present disclosure may be used as a drape for use with an O-ARM multidimensional surgical imaging system, a C-arm multidimensional surgical imaging system, and other surgical imaging system and may be comprised of the following elements. This list of possible constituent elements is intended to be exemplary only, and it is not intended that this list be used to limit the drape of the present application to just these elements. Persons having ordinary skill in the art relevant to the present disclosure may understand there to be equivalent elements that may be substituted within the present disclosure without changing the essential function or operation of the device.

The various elements of the drape system for use with an o-shaped multidimensional surgical imaging system may be related in the following exemplary fashion. It is not intended to limit the scope or nature of the relationships between the various elements and the following examples are presented as illustrative examples only.

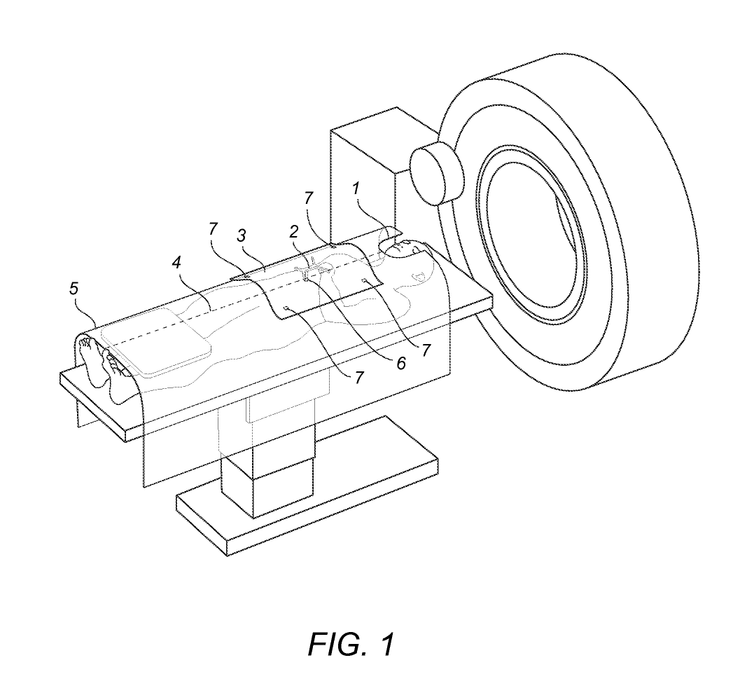

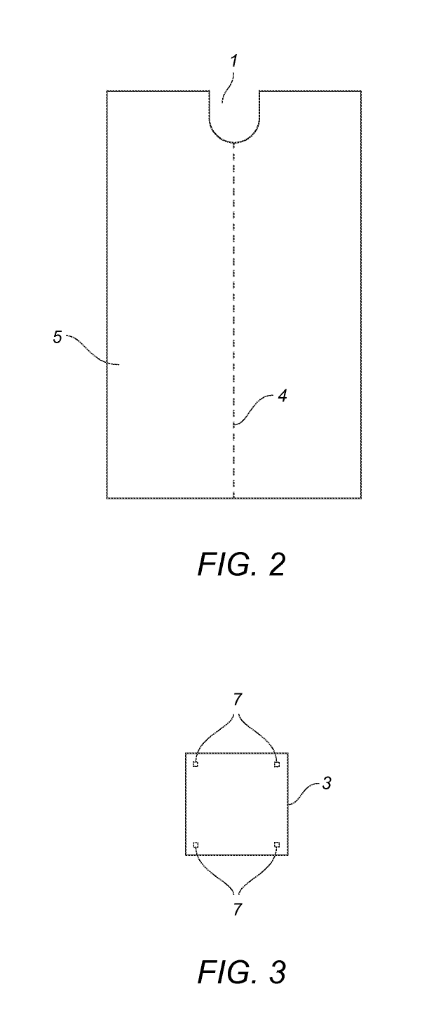

As shown FIGS. 1-3, some embodiments of the invention include a novel device for creating or maintaining a sterile environment when using a multidimensional surgical imagining system, such as the O-ARM multidimensional surgical imaging system or a C-ARM imaging system, the device comprising a drape 5 configured to drape over a patient. The drape 5 may have one or more punctures 6 configured to engage with a spine reference clamp and a reference frame 2. For instance, a spine reference clamp may be connected to a patient in the appropriate location so that the connecting rod from the reference clamp is directed upward away from the patient. The connecting rod from the reference clamp may be inserted through the puncture 6 and the reference frame 2 may then be attached to the end of the connecting rod on the non-patient side of the drape 5, so that the reference frame 2 is not contained within the sterile surgical field underneath the drape 5. That way, the drape 5 may maintain closer contact with the patient without having to hang over the reference frame 2 as well, which may protrude out too far to appropriately cover both the reference frame 2 and the patient with the drape 5. Accordingly, allowing the connecting bar to pass through the puncture 6 will allow the drape 5 to be more securely positioned over the patient. In other embodiments, the drape 5 may hang over the reference frame 2 and not include a puncture 6. The one or more punctures 6 may be positioned along a center line of the drape 5. In other embodiments, the punctures 6 may be positioned at other various locations along the drape 5 to facilitate other reference equipment for assisting an imaging device for surgery.

In embodiments, a removable cover 3 is configured to cover the one or more punctures 6 and the references frame 2 in order to maintain the sterile environment that punctures 6 or other orifices in the drape 5 would otherwise compromise and allow contaminants to pass through. In some embodiments, the cover 3 provides a barrier blocking the puncture 6 to maintain the sterile field between the patient and caregivers. The cover 3 may be attached to the drape 5 using any conventional fasteners and, in some embodiments, is removably attached to the drape 5 using an adhesive attachment 7, as shown in FIG. 1. In some embodiments, adhesive attachment 7 may be four strips of adhesive, for example adhesive tape, that are placed near the four corners of the cover 3. In some embodiments, the cover 3 may include an adhesive strip that is continuous around the perimeter or near the perimeter of the cover 3 in order to provide a seal with the drape 5 or a semi-airtight or watertight seal around the drape 5. In other embodiments, the cover 3 may enclose a reference frame 2 that is attached to a connecting rod of the spinal reference clamp that protrudes through the puncture 6.

As shown in FIG. 1, embodiments of the drape 5 may also comprise a seam 4 positioned along the length of the drape 5, the seam 4 being configured to be pulled apart for removal and disassembly of the drape 5 into separate portions. In some embodiments, the drape 5 may be disposable, and therefore once the seam 4 is pulled apart it for ease of disassembly before discarding. In some embodiments, various reversibly sealable mechanisms may be utilized on the seam 4 in order to seal and unseal the portions of the drape 5 on either side of the seam 4. For instance, in some embodiments, the seam 4 may divide the drape 5 into a first portion and a second portion that when unsealed separates the first and second portions of the drape 5 so that it may be stored, moved, or disposed. Various mechanisms may be utilized for the seam that may include, Velcro, snaps, buttons, zippers, ties, clip and lock components, adhesives, and other suitable attachment methods. In some embodiments, adhesive may be applied to an overlapping section of one of the portions of the drape 5 so that the first and second portions of the drape 5 may be sealed together with adhesive between the overlapping sections.

In some embodiments, the drape 5 may have a cut-out 1 at the end of the drape 5 configured to be proximate to a patient's head. The cut-out may be any shape, such as a U-shape or a squared off shape to prevent the patient's head from being covered by the drape 5, while the remainder of the patient's body is fully covered. Alternatively, the drape 5 may have no cut-out. In these embodiments, the drape 5 is used by pulling the drape 5 up to the patient's shoulders without covering the patient's head or face. Alternatively, if the patient is lying in a prone position (face down), the drape 5 with no cut-out is used by pulling the drape with no cut-out 5 up to the patient's shoulders without covering the patient's head, or up and over the patient's head and covering the patient's head.

In some embodiments, the seam 4 may be partially or fully unsealed to provide an opening that allows a reference clamp to pass through. Once the reference clamp is in place, the seam 4 may be resealed or partially resealed around the reference clamp, for example, by sealing around the reference clamp's connecting bar. If adhesive or other reversibly sealable mechanisms are utilized, the open in the seam 4 may then be close to the maximum extent possible after the reference clamp has passed through. This will allow the seam 4 to perform the function of the puncture 6 as described above. In other embodiments, the seam 4 may be fully re-sealed with both the reference clamp and the reference frame 4 underneath the drape 5. In some embodiments, the seam 4 is also configured to disassemble the drape 5 before, during or after use. For instance, the seam 4 may be unsealed in order to dissemble the drape 5 into separate portions. In some embodiments the seam 4 may separate the drape 5 into two portions, three portions, or more. In one embodiment, the seam 4 may run along the middle of the drape 5 and allow the drape 5 to be disassembled into two relatively equal halves for storage or disposal.

The drape 5 can further comprise a transparent or semi-transparent cover 3 configured to cover the opening in the seam 4, the reference clamp, and a reference frame 2, therefore maintaining a sterile environment. In some embodiments, the cover 3 will be fastened to the drape 5 in four corners using an adhesive or other suitable connection.

In various embodiments, the seam 4 is a fabric hook and loop fastener (e.g., VELCRO® brand fabric hook-and-loop fastener.) In other embodiments, the seam 4 is a ziplock seal. In still other embodiments, seam 4 is joined by an adhesive that allows for unsealing (e.g., allowing for the two sides of the drape to unstick) to allow the reference clamp 2 to pass through or allowing for disassembling the drape 5 after the patient is imaged. The adhesive also allows for resealing (e.g. allowing for the two sides of the drape to re-stick) around the reference clamp.

### Materials for the Drape

Embodiments of the drape may be made of a transparent material, such as the transparent plastic material conventionally used to cover the gantry of O-ARM systems. However, the drape may be made of any other suitable lightweight materials that transmit the appropriate wavelengths of electromagnetic radiation to allow the imaging equipment to detect the reference frame (e.g. the LED, and spheres from the reference frame), or any other conventionally used materials for making surgical drapes.

In some embodiments, the materials for the drape and cover may be impervious to liquid strikethrough which can help to reduce bacterial transfer and subsequent contamination of the surgical site.

Examples of materials that can be used for the drape include but are not limited to polystyrene, polycarbonate, acrylics, silicone rubber, polyethylene, polypropylene, synthetic rubbers, natural fibers, and SMS (spun-melt-spun) fabrics.

Surgical drapes can comprise flexible plastic film, which is adhesive-coated or uses pressure-sensitive adhesive coated tape. The drapes can be in three layers, comprising of a polyurethane film laminated to a releasable layer and a strengthening layer of thicker plastic material, such as polyethylene. Drapes are often designed to soak up spills and splatters while not allowing fluid strike-through.

Another example of appropriate materials is the material used in 3M™ Steri-Drape™ 9000 Surgical Drapes which is a laminate of polypropylene fibers and a polyethylene film.

### Sizes

The drape may be substantially rectangular, circular, or any other suitable shape. In some embodiments, the drape may have any suitable size that covers the entire patient, or a patient's body. The size can be adapted for the type of subject (e.g., human, adult, child, veterinary). For example, some drapes may be about 5 feet wide. Other widths can include about 2, 3, 4, 5, 6, 7, 8 feet. The drapes may have lengths of, for example, about 6 feet, about 7 feet, or about 8 feet. Additional examples of lengths can be about 3, 4, 5, 6, 7, 8, 9, 10 feet. In fact, the drape may have any width and length depending on the size of the patient. In some embodiments, the drape may be 72″L×60″W, 72″L×60″W, 84″L×60″W, or 96″L×60″W.

In some embodiments, the drape can be long enough and wide enough to cover stands (e.g., mayo stand) and instrumentations attached to the surgery table. In particular embodiments, the drape is sized so that when in place over the patient, it does not touch the floor based on the height of the operating table.

### Head Cut-Out

The cut-out may be about 12 inches wide and about 12 inches long. Other cut-out sizes can be about 9, 10, 11, 12, 13, 14, 15 inches wide and about 9, 2, 10, 11, 12, 13, 14, 15, inches long.

### Puncture

In various embodiments, the drape may include a puncture for a spine reference clamp or other surgical imaging reference tool. The puncture would allow a connecting rod from a spine reference clamp to pass through the drape so that the reference frame could be attached to the side of the drape opposite the patient. Accordingly, the puncture may be round and about 1 inch in diameter to accommodate a bar from a spine clamp. However, the puncture may have any necessary shape and size and located at any appropriate point on the drape. For instance, in some embodiments, the puncture may be located anywhere along an approximately center line of the length of the drape. This may be preferable when the drape is used for spinal surgeries. In instances wherein the drape is used for orthopedic surgeries, the puncture may be along on an approximately center line of the length of the drape. In some embodiments, the puncture may be just offset to the side from the center line to avoid interference with the seam. In other instances, the puncture may be on a different location of the drape. For example, in an upper right quadrant for right shoulder surgeries, in a lower left quadrant for left knee surgeries; or in an off center position for a hip surgery.

The puncture can also be configured to be expanded to allow the reference frame to fit through. In one example, the puncture can be manually torn further to expand the size of the puncture. In another example, the puncture can have resealable seams that extend from it to allow for the expansion of the puncture and resealing of the expanded puncture to fit around the reference clamp.

### Cover

In various embodiments, the cover may be at least partially transparent to the visible light spectrum, such that a user can see the puncture and the reference frame on the drape through the cover. Additionally, the cover may also pass electromagnetic radiation with wavelengths in the range of those emitted from the reference frame to allow the imaging system to detect the emissions (e.g., LED, sphere, halogen) from the reference frame. The cover may be made of any material that allows it to be relatively transparent and preferably flexible such that it can be used to cover the reference frame.

In certain embodiments, the cover may be removably attached to the drape using any conventional fasteners, such as adhesive tape. Other examples of fasteners include but are not limited a hook-and-loop fastener (also known as touch fastener) (e.g., VELCRO® brand hook-and-loop fastener or touch fastener), a ziplock seal (e.g., ZIPLOC® brand ziplock seal), a button, a snap fastener, a clasp, a hook and eye closure, tie, twist tie, or a zipper.

In certain embodiments, a first portion of the cover is attached to the drape and a second portion of the cover is configured to be removably attached to the drape. For example, one side of the cover may be permanently attached to the drape and create a flap that covers a puncture and reference frame. In this embodiment, after the drape is placed over the patient the reference clamp, and potentially the reference frame may be put into place by passing them through the puncture with the flap open to allow access through the puncture. In other embodiments, the reference clamp may be first put into place, and then the drape may be placed over the reference clamp. In this embodiment, the connecting rod from the reference clamp could then be passed through the puncture and connected to the reference frame. Then, after the claim and frame are in place, the flap can be placed over the puncture, and the reference clamp and reference frame and the other side(s) of the cover can be removably attached to the drape with a fastener. Accordingly, a sterile field will be maintained.

In various embodiments, the cover may be substantially square in shape and have a length of about 2 feet and a width of about 2 feet. However, other shapes and dimensions may be used as well; for example, circular, rectangular, triangular shapes, and dimensions of about 1, 2, 3 feet length by about 1, 2, 3 feet width. In some embodiments, the cover 3 may be constructed from lightweight material to allow for easy removal and closure of the cover 3. In some embodiments, the cover 3 may be made with plastic that has a small enough gauge to allow it to maintain good contact with bulbs of a reference frame when laid over the top of the reference frame 2. For instance, the cover may be a plastic with a gauge of 0.005, 0.010, 0.020, or 0.030, 0.10, 0.20, 0.2 inches or other thicknesses.

### Methods

Various embodiments of the present invention provide for methods of securing the sterile field for a subject during intraoperative imaging. In various embodiments, the method comprises providing a device, comprising a drape configured for covering a patient, one or more punctures on the drape configured to allow a reference clamp to pass through the drape, and a resealable seam on the drape configured to disassemble the drape into at least two separate portions before, during or after use; placing the device over the patient, wherein a reference clamp and/or a reference frame is passed through the one or more punctures; securing the cover over the one or more punctures, the reference clamp, and/or reference frame. The method can further comprise removing the device after the imaging is performed by pulling apart the seam on the drape.

In various embodiments, the method comprises providing a device, comprising: a drape configured for draping over a subject; and a resealable seam on the drape configured to open and allow a reference clamp to pass through the drape and configured to disassemble the drape before, during or after use; placing the device over the subject; unsealing a portion of the seam to allow a reference clamp and/or a reference frame to pass through the drape; and securing a transparent cover over the reference clamp, and/or reference frame.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A device, comprising:
a drape configured for draping over a subject, the drape comprising a first portion and a second portion;
one or more punctures in the drape sized to allow at least a portion of a reference clamp to pass through the puncture;
a resealable seam connecting the first and second portions of the drape; and
a cover configured to removably obstruct the one or more punctures, the cover configured to allow electromagnetic radiation to pass through it.

2. The device of claim 1, wherein the one or more punctures is configured to also allow a reference frame to pass through.

3. The device of claim 1, wherein the cover reversibly slides to obstruct the cover on a pair of rails connected to the drape.

4. The device of claim 1, wherein the resealable seam comprises a fabric hook and loop fastener.

5. The device of claim 1, wherein the resealable seam comprises a ziplock seal.

6. The device of claim 1, wherein the resealable seam comprises an adhesive.

7. The device of claim 1, wherein the cover is configured to be removably attached to the drape.

8. The device of claim 1, wherein the electromagnetic radiation comprises wavelengths in the range emitted by an LED or a halogen lamp.

9. The device of claim 1, wherein the resealable seam includes a fastener comprising an adhesive fastener, a hook-and-loop fastener, a ziplock seal, a button, a snap fastener, a clasp, a hook and eye closure, tie, twist tie, or a zipper.

10. The device of claim 1, wherein a first portion of the cover is attached to the drape and a second portion of the cover is configured to be removably attached to the drape.

11. The device of claim 10, wherein the second portion of the cover is configured to be removably attached to the drape with a fastener.

12. A device, comprising:
a drape configured to cover a patient;
a seam on the drape configured to reversibly seal at least a first and second portion of the drape together;
wherein at least a portion of the seam is configured to unseal to allow at least a portion of a reference clamp to pass through; and
a cover configured to cover the opening in the resealable seam, the reference clamp, and a reference frame.

13. The device of claim 12, wherein the resealable seam comprises a fabric hook and loop fastener.

14. The device of claim 12, wherein the resealable seam comprises a ziplock seal.

15. The device of claim 12, wherein the resealable seam comprises an adhesive.

16. The device of claim 12, wherein the cover is configured to be removably attached to the drape.

17. The device of claim 16, wherein the cover is removably attached to the drape with a fastener.

18. The device of claim 17, wherein the fastener is an adhesive fastener, a hook-and-loop fastener, a ziplock seal, a button, a snap fastener, a clasp, a hook and eye closure, tie, twist tie, or a zipper.

19. The device of claim 12, wherein a first portion of the cover is attached to the drape and a second portion of the cover is configured to be removably attached to the drape.

20. The device of claim 19, wherein the second portion of the cover is configured to be removably attached to the drape with a fastener.

21. The device of claim 20, wherein the fastener is an adhesive fastener, a hook-and-loop fastener, a ziplock seal, a button, a snap fastener, a clasp, a hook and eye closure, tie, twist tie, or a zipper.

22. A method for creating a sterile environment during intraoperative imaging, comprising:
providing a device of claim 1 or a device comprising,
a drape configured to cover a patient;
a seam on the drape configured to reversibly seal at least a first and second portion of the drape together;
wherein at least a portion of the seam is configured to unseal to allow at least a portion of a reference clamp to pass through; and
a cover configured to cover the opening in the resealable seam, the reference clamp, and a reference frame;
covering the subject with the device; and
placing the cover over the reference frame.

23. The method of claim 22, further comprising removing the device from covering the patient by unsealing the resealable seam.

24. A device for creating a sterile environment isolated from an o-shaped or c-shaped multidimensional surgical imaging system, the device comprising:
a drape sized to cover a patient, the drape comprising:
a puncture configured to engage with a spine reference clamp, the puncture positioned along a center line of the drape;
a reference frame connected to the spine reference clamp, wherein each of the puncture, the spine reference clamp, and the reference frame are positioned along the center line of the drape; and
an adhesive seam that removably connects at least a first and second portion of the drape; and
a cover removably attached to the drape, the cover being configured to cover the puncture and the reference frame.
